SEQUENCE LISTING (29 sequences)

SEQ ID NO: 1 - DNA, 43 bp, synthetic construct
  source 1..43: mol_type = other DNA
ttaagatacattgatgagtttggacaaaccacaactagaatgc

SEQ ID NO: 2 - DNA, 23 bp, synthetic construct
  source 1..23: mol_type = other DNA
agtaggagcaatcacaggtgagc

SEQ ID NO: 3 - DNA, 42 bp, synthetic construct
  source 1..42: mol_type = other DNA
gtacgcgtatcgataagctttaagatacattgatgagtttgg

SEQ ID NO: 4 - DNA, 28 bp, synthetic construct
  source 1..28: mol_type = other DNA
taggggccgcgactctagatcataatca

SEQ ID NO: 5 - DNA, 31 bp, synthetic construct
  source 1..31: mol_type = other DNA
ctgcggcgatcgaaaggcaagggcattcagc

SEQ ID NO: 6 - DNA, 23 bp, synthetic construct
  source 1..23: mol_type = other DNA
atatgcgagctcgcccggggatc

SEQ ID NO: 7 - DNA, 24 bp, synthetic construct
  source 1..24: mol_type = other DNA
tgcatcgaattggtcggactggtc

SEQ ID NO: 8 - DNA, 54 bp, synthetic construct
  source 1..54: mol_type = other DNA
cttatcgatacgcgtacgctagcgacgtcttccagtgtccaaaacccacagccg

SEQ ID NO: 9 - DNA, 27 bp, synthetic construct
  source 1..27: mol_type = other DNA
ctaggccgtgggcatcggcaataccac

SEQ ID NO: 10 - DNA, 24 bp, synthetic construct
  source 1..24: mol_type = other DNA
cgctagcgtacgcgtatcgataag

SEQ ID NO: 11 - DNA, 24 bp, synthetic construct
  source 1..24: mol_type = other DNA
cctaggcgagctcgcccggggatc

SEQ ID NO: 12 - DNA, 50 bp, synthetic construct
  source 1..50: mol_type = other DNA
tccccgggcgagctcgcctaggttttttgctcacctgtgattgctcctac

SEQ ID NO: 13 - DNA, 45 bp, synthetic construct
  source 1..45: mol_type = other DNA
attgccgatgcccacggcctaggacaaaagctggagctcctgcag

SEQ ID NO: 14 - DNA, 60 bp, synthetic construct
  source 1..60: mol_type = other DNA
ctcacctgtgattgctcctacttttcgtttttttgctttcgccagtatttattatttttc

SEQ ID NO: 15 - DNA, 55 bp, synthetic construct
  source 1..55: mol_type = other DNA
gatctagagtcgcggcccctattacttatacagttcatccattcccatcacgtcg

SEQ ID NO: 16 - DNA, 30 bp, synthetic construct
  source 1..30: mol_type = other DNA
gccaccatggtgtcgaagggcgaggaggac

SEQ ID NO: 17 - DNA, 26 bp, synthetic construct
  source 1..26: mol_type = other DNA
cttcgacaccatggtggcgaccggtg

SEQ ID NO: 18 - DNA, 60 bp, synthetic construct
  source 1..60: mol_type = other DNA
ctttcgatcgccgcagacgtcgtaaggctagttttgagaaggatcgcttgtctgggcaag

SEQ ID NO: 19 - DNA, 60 bp, synthetic construct
  source 1..60: mol_type = other DNA
atgtatcttaaagcttatcgatacgcgtacgctagccaaggtgctacgaaatccgttgtg

SEQ ID NO: 20 - DNA, 664 bp, synthetic construct
  source 1..664: mol_type = other DNA
gttcacaacggatttcgtagcaccttggctagcggctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggcatccaagtatcgccatcgttttagagctagaaatagcaagttaaaataaggctagtccgttatcaacttgaaaaagtggcaccgagtcggtgctttttttgcctacctggagcctgagagttgttcaataaaataaaaatgtttcgtttttttgctttcgccagtatttattatttttcatcaatatgtattcgctagcgtacgcgtatcgataagctttaagatac

SEQ ID NO: 21 - DNA, 44 bp, synthetic construct
  source 1..44: mol_type = other DNA
acgtcgtaaggctagttttgagaaggatcgcttgtctgggcaag

SEQ ID NO: 22 - DNA, 60 bp, synthetic construct
  source 1..60: mol_type = other DNA
ctcaaaactagccttacgacgtggcgatacttggatgccctgcggcgatcgaaaggcaag

SEQ ID NO: 23 - DNA, 45 bp, synthetic construct
  source 1..45: mol_type = other DNA
atccccgggcgagctcgcatatatccgggatgcgactgctcaatg

SEQ ID NO: 24 - DNA, 20 bp, synthetic construct
  source 1..20: mol_type = other DNA
ggcgatacttggatgccctg

SEQ ID NO: 25 - DNA, 20 bp, synthetic construct
  source 1..20: mol_type = other DNA
ggttttggacactggaaccg

SEQ ID NO: 26 - DNA, 20 bp, synthetic construct
  source 1..20: mol_type = other DNA
gccacaattgtcgatcgtca

SEQ ID NO: 27 - DNA, 20 bp, synthetic construct
  source 1..20: mol_type = other DNA
ggcatccaagtatcgccatc

SEQ ID NO: 28 - DNA, 29 bp, synthetic construct
  source 1..29: mol_type = other DNA
ccggatggcgatacttggatgccctgcgg

SEQ ID NO: 29 - DNA, 29 bp, synthetic construct
  source 1..29: mol_type = other DNA
ccgcagggcatccaagtatcgccatccgg

(19) United States
(12) Patent Application Publication
(10) Pub. No.: US 2024/0392287 A1
(43) Pub. Date: Nov. 28, 2024

(54) METHODS FOR DELAYING CRISPR ACTION AND IMPROVING GENE DRIVE EFFECTIVENESS

(71) Applicant: The Regents of the University of California, Oakland, CA (US)

(72) Inventors: Valentino Matteo GANTZ, La Jolla, CA (US); Alena Lauren BISHOP, Berkeley, CA (US); Victor LOPEZ DEL AMO, Pearland, TX (US)

(73) Assignee: The Regents of the University of California, Oakland, CA (US)

(21) Appl. No.: 18/695,158

(22) PCT Filed: Oct. 4, 2022
(86) PCT No.: PCT/US2022/077504

§ 371 Date: Mar. 25, 2024

Related U.S. Application Data
(60) Provisional application No. 63/253,639, filed on Oct. 8, 2021.

(51) Int. Cl. C12N 15/11; A61K 48/00; C12N 9/22; C12N 15/82; C12N 15/90

(52) U.S. Cl. CPC C12N 15/111; A61K 48/005; C12N 9/22; C12N 15/8201; C12N 15/907; C12N 2310/20

(57) ABSTRACT

The present disclosure provides the ability of low-cutting gRNAs to circumvent the maternal effect by delaying their action in time. Low-cutting gRNAs lead to a compounding dynamic drive efficiency, which results in drive efficiencies comparable to that of high-cutting gRNAs. Additionally, a gene drive system with a low-cutting gRNA outperforms a more efficient gRNA in laboratory caged populations.

### CROSS-REFERENCE

This application claims the benefit of U.S. Provisional Application No. 63/253,639, filed Oct. 8, 2021, the entire content of which is incorporated herein by reference.

### STATEMENT REGARDING FEDERALLY SPONSORED RESEARCH OR DEVELOPMENT

This invention was made with government support under grant number OD023098 and AI162911 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Oct. 3, 2022, is named 942103-2110_SL.xml and is 27,468 bytes in size.

### FIELD AND BACKGROUND

The present disclosure relates to improve effectiveness of CRISPR gene drive systems and/or technologies.

Gene-drive systems can propagate engineered traits through populations to help curb the impact of vector-borne diseases¹⁻³. For example, mosquitos can be engineered to carry genes that suppress either the vectored pathogens⁴⁻⁶ or the disease vector populations⁷⁻⁹. As such, these tools offer potential solutions for crop pest control¹⁰ and invasive species population suppression^{11,12}, such as for rodent species^{13,14}, which currently impact island conservation campaigns¹⁵. While novel gene-drive technologies have bloomed after the advent of CRISPR, the technology is still in its infancy and has major issues that need to be addressed before its universal application in the wild.

Briefly, CRISPR gene drives can bias their inheritance from Mendelian (˜50%) to super-Mendelian (>50%) rates by converting heterozygous germline cells to homozygosity. A basic gene-drive construct encodes both a Cas9 endonuclease and a guide RNA (gRNA) that targets the precise location chosen for insertion of the gene-drive transgene into the genome. When a gene-drive individual mates with a wildtype counterpart, in the resulting offspring the Cas9/gRNA complex cleaves the wildtype allele that opposes the gene drive. Subsequently, the endogenous homology-directed DNA-repair (HDR) machinery mends the double-stranded DNA break by copying the gene-drive sequences from the gene-drive chromosome onto that of the cleaved wildtype one^{16,17}. When this process occurs in the germline, the gene-drive inheritance is positively biased, allowing the engineered allele to propagate through a population.

The CRISPR system has two components (Cas9 and gRNA) that can be used to generate split, gRNA-only gene-drive systems (e.g., CopyCat elements), which contain only a gRNA-expressing gene inserted at the gRNA-targeted location^{13,17,18}. These elements can be combined with a Cas9-expressing transgene to activate the gene-drive process. Split drives, such as CopyCats, can increase flexibility and permit the evaluation of specific component variables by selective alteration of just one of the two transgenes¹⁹. Within these systems, the gene-drive field has mainly focused on achieving efficient HDR-mediated, allelic conversion by optimization of individual gene-drive elements for their highest efficiency^{19,20}. Factors such as the gRNA target^{18,21}, the promoter selected for Cas9 expression^{19,21,22}, and homology at the targeted locus¹⁹, can influence gene-drive performance.

While different gene drives have shown consistent efficiency when inherited from a father, inheritance from a female causes a maternal effect that can negatively impact the gene-drive process. It is thought that the mother deposits Cas9/gRNA complexes in the egg that lead to a premature cleaving of the incoming paternal allele. At this time in development, error-prone DNA-repair pathways efficiently generate small mutations at the target site that prevent further gene-drive propagation^{4,19}. This effect has been described in Drosophila melanogaster^{19,21} and mosquitoes 4. Different groups have attempted to resolve it through: 1) identification of different promoters for Cas9 expression to better restrict transcription to the developing germline^{19,22}; or 2) supplementation of the gene drive with a re-coded version of the disrupted gene so non-conversion events would yield mutant individuals with lower fitness, such that the maternal effect occurs without affecting gene-drive spread through selective pressure^{5,23}. While there has been some success with these methods, there is still no clear-cut resolution to the maternal effect. The mechanism of the maternal effect was previously characterized in detail and its cause to the premature cutting by the Cas9/gRNA deposited in the early embryo before germline determination was pinpointed¹⁹, which occurs in Drosophila melanogaster at ˜1.5 hours after fertilization²⁴. It was suggested that gene-drive strategies that delayed cutting during the first hours of development should bypass the maternal effect, and lead to a highly efficient gene drive that would be similar to paternal inheritance¹⁹.

### SUMMARY

The present disclosure provides a method of using low efficiency gRNAs to bypass a maternal effect that can impair the successful application of CRISPR-based gene drive technologies. In certain embodiments, the method provided in the present disclosure uses low-efficiency gRNAs to circumvent the maternal effect by delaying the gRNA action in time. Additionally, these low-cutting gRNAs lead to a dynamic drive efficiency that compounds over time and results in drive efficiencies comparable to high-cutting gRNAs, making them a better option for gene drive. In our work we show that a gene drive system using a low-efficiency gRNA outperforms one carrying an efficient gRNA in cage populations.

In certain embodiments, four gRNA-only drives, each carrying gRNAs of different efficiencies were evaluated, and their effectiveness in bypassing the maternal effect affecting gene drive were measured. It was found that lower efficiency gRNAs can bypass the maternal effect by delaying target cleavage to later in development, effectively delaying drive activity beyond the critical time for the maternal effect. Such low-efficiency gRNAs are also capable of continuing gene-drive conversion in the adult germline to achieve, as the flies age, similar inheritance rates as those produced by high-cutter gRNAs. Moreover, the spread of a high-cutter and a low-cutting gRNA were also evaluated in laboratory caged populations, and it was showed that the latter has a consistently higher gene drive performance. Overall, the move from the field-standard high-efficiency agents to a low-efficiency gRNA has overcome the gene-drive-killing maternal effect in insects, which should pave the way for more effective applications of gene drives.

Beyond the use for gene drive, low cutting gRNAs could be similarly used to delay action in time. This feature is sought in diverse systems such as human cells for therapy (e.g., long or delayed action by CRISPR), or in agriculture to have CRISPR act at different developmental stages, such as pollen fruit or flower formation.

Therefore, the present disclosure provides a method to improve gene drive performance when using this method to modify or suppress wild populations of insects such as flies, mosquitoes or other insects. The same strategy could be used also in other animals or plants beyond the order diptera (true flies). Further, the method of the present disclosure could be used to delay action in time of the CRISPR components in other situations, and in systems different from insects (e.g., plants, mammals) for diverse applications. Human therapy might need long or delayed action by CRISPR, to ensure a gradual or timely delivery of genetic changes. In agriculture, the method of the present disclosure could help to have CRISPR act at different plant developmental stages, such as pollen fruit or flower formation, where the genetic edits might be more beneficial or effective.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Many aspects of the present disclosure can be better understood with reference to the drawings. The components in the drawings are not necessarily to scale, emphasis instead being placed upon clearly illustrating the principles of the present disclosure. Moreover, in the drawings, like reference numerals designate corresponding parts throughout the several views. A better understanding of the features and advantages of the invention will be obtained by reference to the following detailed description that sets forth illustrative embodiments, in which the principles of the invention are utilized, and the accompanying drawings (also “Figure” and “FIG.” herein), of which:

FIGS. 1a-1d. Engineering gene-drive systems with different conversion efficiencies. FIG. 1a. Sequences targeted by the w2, y1, e1, and w5 gRNAs. Cut site is marked by a vertical line; PAM is in red. The efficiencies as predicted by the CHOPCHOP tool are noted on the right side of each sequence. Figure discloses SEQ ID NOS 24-27, respectively in order of appearance. FIG. 1a′. Relative positioning of the w2- and w5-gRNA targets. Figure discloses SEQ ID NOS 28-29, respectively in order of appearance. FIG. 1b. Constructs used in this manuscript. FIG. 1c. Cross scheme used in these experiments. A Cas9-expressing male is crossed with a drive-containing female. The resultant heterozygous females are outcrossed in single pairs and the F₂ phenotypes are scored. FIG. 1d. Results from the crosses outlined in FIG. 1c for CopyCat drives at w2, y1, e1, and w5. Average inheritance of the drive, standard deviation, and number of crosses and flies scored are denoted.

FIGS. 2a-2f. Inheritance of gene-drive constructs with different gRNA cutting efficiencies. FIG. 2a. Cross scheme to examine paternal inheritance. A male fly containing both the Cas9 and drive transgenes is crossed to a wildtype female. The resulting heterozygous females are outcrossed in single pairs for 5 days, passed to a new vial for days 6-10, then passed to a third vial for days 11-15. F₂ progeny from each of the three vials are scored separately. FIG. 2b Same as FIG. 2a, except an F₀ female containing both the Cas9 and drive transgenes is crossed to a wildtype male to examine maternal inheritance. FIGS. 2c-2f. Results from paternal inheritance crosses (left) and maternal inheritance crosses (right) for the FIG. 2c w2, FIG. 2d y1, FIG. 2e e1, and FIG. 2f w5 drives. The results from days 1-5 are the first column in each section, followed by days 6-10 and days 11-15. Examples of a single cross from which F₂ flies were scored at each timepoint are shown by dots circled in black and connected by a dashed line. FIG. 2e,2f. In these experiments, we observe two different conditions: when the inheritance from single F₁ flies increased over time, the dotted line is displayed in green; while when the maternal effect is present and the inheritance does not increase over time, the dotted line is colored in red. This latter conclusion is further supported by the presence of ˜50% of a specific mutant phenotype (ebony- or white-) in the recorded F₂ phenotypes of crosses displaying maternal effects.

FIGS. 3a-3c. Analysis of the spread of w2- and w5-gRNA CopyCat elements in caged populations. FIG. 3a. Schematic of the yellow and white genes, located on the X chromosome. A Cas9 transgene is inserted at the yellow locus. The w2/w5 target sites are labeled. The w^{-EX1} mutation, in the first exon of white (denoted with *) is ˜3.6 kb away from the gRNA cut sites. FIG. 3b. Schematic of the bottle handling protocol. Bottles are seeded with 100 flies, with genotypes indicated on the left. After five days, adult flies are removed, and the remaining eggs and larvae are allowed to develop until day 18. Flies are then phenotypically scored and 200 of them, in a ratio representative of the population, are used to seed the next generation. The process is then repeated. FIG. 3c. Results from w2 and w5 drives spreading in a population for 12 generations, done in triplicate. Dashed lines represent individual cages, and the average of the three replicates is represented by the solid line.

FIGS. 4a-4d. FIG. 4. Model explaining the behavior of the w2- and w5-gRNA CopyCat elements. FIG. 4a. Model of the w2 drive with paternal inheritance. Cas9 protein is absent from the egg and cutting is delayed until the germline development stage (marked by a red bar), resulting in efficient conversion by HDR and high drive inheritance rates. FIG. 4b. Model of the w2 drive with maternal inheritance. Here, Cas9 protein is present in the egg, leading to early cutting (red bar) and low HDR rates, resulting in a high prevalence of resistant alleles. FIG. 4c. Model of the w5 drive with paternal inheritance. Cas9 protein is absent from the egg, and the gRNA has lower efficiency than w2. This delays cutting to later stages of development (red-gradient bar), resulting in modest drive inheritance rates. As time passes, however, more and more of the germlines have a chance to be converted (bottom panel). FIG. 4d. Model of the w5 drive with maternal inheritance. Cas9 protein is present in the egg, but because of the lower efficiency of the w5 gRNA, cutting is still delayed and can happen throughout development (red dashes). The maternal effect is mostly avoided, and as time passes more of the resulting germlines have a chance to be converted by HDR (bottom panel).

FIGS. 5a-5i. Detailed cross schemes used to evaluate gene drive in this manuscript. FIGS. 5a,5b,5c. Genetic crosses used in FIGS. 1a-d. FIGS. 5d, 5e, 5f. Genetic crosses to assess paternal inheritance used in FIGS. 2a-2f. FIGS. 5g, 5h, 5i. Genetic crosses to assess maternal inheritance used in FIGS. 2a-2f.

FIG. 6. Low-efficiency gRNA have low targeting and leave alleles uncut. Proportions of F₂ flies from crosses in FIGS. 1a-1d that either inherited the drive element (CopyCat), had an indel resulting in the recessive phenotype (Mutant), or had the wildtype phenotype. The wildtype phenotype would indicate an untouched allele or an indel that has been repaired in frame, yielding a functional gene.

FIGS. 7a-7c. Evaluation of conversion in the male germline. FIG. 7a. Schematic of cross to examine inheritance of the e1 drive through an F₁ male with the drive element coming from the F₀ male and Cas9 coming from the F₀ female. Heterozygous F₁ males were then crossed in single pairs to females with an ebony deletion and F₂ flies were scored for body color and fluorescence. FIG. 7b. Schematic of cross to examine the maternal inheritance of the e1 drive through an F₁ male. An Fo female containing both the drive and Cas9 elements was crossed to a wildtype male. Heterozygous F₁ males were single-pair crossed to females with an ebony deletion for 5 days, then the adults were passed to a new vial for days 6-10 and another new vial for days 11-15. F₂ flies from each of the three timepoints were scored separately for body color and fluorescence. FIG. 7c. Drive inheritance results from the crosses outlined in (FIG. 7a) and (FIG. 7b).

FIGS. 8a-8b. Evaluating different efficiency gene drives' spread in caged populations with an alternative protocol. FIG. 8a. Schematic of the cage protocol using random sampling. Bottles were seeded with 100 flies total, genotypes listed on the left, and left for 5 days. Adult flies were then discarded and the remaining eggs and larvae allowed to develop until day 18. Approximately 200 of these new flies were then randomly selected to seed the next generation while all remaining flies were scored for fluorescence. The process was then repeated. FIG. 8b. Results tracking the spread of w2 and w5 drives in caged populations for 14 generations using the protocol outlined in (FIG. 8a). Each condition was done in triplicate; dashed lines represent individual cages and solid lines represent the average of the three replicates.

FIGS. 9a-9c. Evaluating different efficiency gene drives' spread in overlapping generation caged populations. FIG. 9a. Photos of the cages used for these experiments. There are slots for 4 food vials to be inserted and later exchanged without flies escaping. Cages walls are made out of acrylic sheets and have vents covered with wire mesh for air flow. The white pieces used to exchange vials are 3D-printed using PLA plastic. FIG. 9b. Schedule on which food vials were exchanged and flies were scored. A, B, C, D represent the four slots in which vials can be placed. Vials are left for one day in slot A before being removed and any eggs deposited in the food are allowed to develop, separate from the cage. Every third vial is eventually returned to the cage to maintain the population; before flies eclose (9 days after vial was inserted), the vial is returned to another slot (B or C) and flies can emerge into the cage for 6 days (up to day 18). All other vials are not returned to the cage and are instead scored for fluorescence on day 18. FIG. 9c. Results from w2 and w5 drives spreading in continuous population cages. Dashed lines follow actual data points, the small solid lines represent the 12-day averages, and the bigger lines represent the 10-day moving averages of the average.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a catalyst,” “a metal,” or “a substrate,” includes, but are not limited to, mixtures or combinations of two or more such catalysts, metals, or substrates, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Unless otherwise specified, temperatures referred to herein are based on atmospheric pressure (i.e., one atmosphere).

In certain embodiments, the present disclosure provides a method for delaying CRISPR action and improve gene drive effectiveness.

In certain embodiments, the present disclosure provides that gene drives built using low-cutting gRNAs can bypass the drive-killing maternal effect caused by the deposition of Cas9/gRNA complexes in insect eggs, which has hampered the application of gene drives, and caused concern regarding their effectiveness during an eventual field deployment. The lower activity of these gRNAs delays target cleavage until later in development when HDR is the predominant DNA-repair mechanism, effectively bypassing the developmental stage wherein the maternal effect is most detrimental to gene-drive spread. Since these gRNA do not fully target all the cells of a fly after reaching adulthood, HDR-mediated allelic conversion keeps acting in the adult stages, resulting in progressively increasing gene-drive inheritance as the flies age. Further, it was shown that w5, a low-cutting gRNA, outperforms the high-cutter w2-gRNA in laboratory caged populations, consistently reaching higher gene-drive-allele frequency. To enable this analysis, ad-hoc-designed, large cages were designed that allow for a low-effort maintenance of large overlapping-generation laboratory populations without compromising the safety standards required for gene-drive work.

These findings that for some applications, such as gene drives in insects, the use of low-efficiency gRNAs bypasses the maternal effect observed in different organisms^{4,5,19} for better overall performance goes against the trends in the gene-drive field; since the field has so-far focused on maximizing the efficiency of the various drive components, this shift to low-efficiency components represents a significant paradigm change. Several groups have also attempted to modulate the expression of Cas9 to avoid CRISPR component deposition in the egg^{19,22}, though the potential use of low-efficiency gRNAs is a far simpler method for achieving this goal. While the identification of promoters with expression exquisitely restricted to the germline is challenging, the efficiency of gRNAs can be predicted bioinformatically²⁵ and easily and quickly tested in cells or in vivo²⁰.

Besides circumventing the maternal effect, the use of low-efficiency gRNAs could be also employed in other situations where delayed action of gRNAs may be required. In mice, for example, when a gene drive is built using a strong ubiquitous promoter, efficient, early activity in the very first embryonal cell divisions leads to the generation of resistant alleles that inhibit further conversion¹³; low-efficiency gRNAs could be employed in such a system to delay cutting and perhaps generate gene drives using these promoters, which could be more effective than germline ones^{13,14}. Separately, genetic sterile-insect-technique strategies based on CRISPR rely on the combination of Cas9 and gRNA individuals to generate sterile males for field release²⁶. This process requires large numbers of individuals to be collected for genetic crosses, which might be difficult to scale for commercial purposes. Low-efficiency gRNAs allows one to combine the Cas9 and gRNA transgenes in a single viable line, which when aged, allows for the collection of solely sterile males.

The present disclosure further provides that the disclosed method for delaying CRISPR action and improve gene drive effectiveness has technical implications for the gene-drive field, as small differences in protocols could have dramatic effects in the interpretation of data. For example, gene-drive virgin flies were collected and they were crossed the day after eclosion and four days thereafter; collecting several virgins over a span of a few days and later setting up crosses at the same time is common practice of Drosophilists. In the case of w5, the latter approach could generate very different results than a more rigorous protocol and might result in similar gene-drive levels as w2. While other insects like mosquitoes differ in development timeline, it is possible that insect age or other protocol aspects could severely impact the interpretation of the data.

Overall, the present disclosure provides that low-efficiency gRNAs can be successfully employed to generate efficient gene-drive systems that bypass the maternal effect, offering a dramatically new direction for mosquito work that has been previously focused on maximizing gRNA efficiency. The continued gene-drive conversion from low-cutting gRNAs during the lifetime of an individual could make this phenomenon more pronounced, leading to much improved systems. This is especially true in mosquitoes, which in all likelihood do not readily mate and lay eggs after eclosion in the wild, and this could be tested by performing a similar analysis on mosquitoes of different ages, or in large-population simulated-environment experiments. Altogether, the present disclosure provides a paradigm-shifting work highlighted the potential of low-cutting gRNAs as a viable option for gene-drive construction and could impact the design and strategies for field applications of these technologies.

Now having described the aspects of the present disclosure, in general, the following Examples describe some additional and/or more detailed aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Methods and Materials

### Plasmid Construction

All plasmids were built using standard molecular biology techniques. Plasmids were constructed by Gibson Assembly using NEBuilder HiFi DNA Assembly Master Mix (New England BioLabs Cat. #E2621) and transformed into NEB 10-beta electrocompetent E. coli (New England BioLabs Cat. #3020). Plasmids were purified using a Qiagen Plasmid Midi Kit (Qiagen Cat. #12143) and sequences were confirmed by Sanger sequencing at Genewiz. Primers used for cloning and further detail for each construct can be found in the following Table.

| | SEQ ID | |
|---|---|---|
| Name | NO: | Primer sequence |
| 279 | 1 | TTAAGATACATTGATGAGTTTGGACAAACCACAACTAGAATGC |
| 440 | 2 | AGTAGGAGCAATCACAGGTGAGC |
| 451 | 3 | GTACGCGTATCGATAAGCTTtaaGATACATTGATGAGTTTGG |
| 484 | 4 | TAGgggccGCGACTCTAGATCATAATCA |
| 611 | 5 | CTGCGGCGATCGAAAGGCAAGGGCATTCAGC |
| 1068 | 6 | atatgCGAGCTCGCCCGGGGATC |
| 1119 | 7 | TGCATCGAATTGGTCGGACTGGTC |
| v282 | 8 | CTTATCGATACGCGTACgctagcgacgtcttccagtgtccaaaacccacagccg |
| v283 | 9 | CTAGGCCGTGGGCATCGGCAATACCAC |
| v284 | 10 | cgctagcGTACGCGTATCGATAAG |
| v285 | 11 | CCTAGGCGAGCTCGCCCGGGGATC |
| v286 | 12 | TCCCCGGGCGAGCTCGCCTAGGTTTTTTGCTCACCTGTGATTGCTCCTAC |
| v287 | 13 | ATTGCCGATGCCCACGGCCTAGGACAAAAGCTGGAGCTCCTGCAG |
| v301 | 14 | CTCACCTGTGATTGCTCCTactTTTCGTTTTTTTGCTTTCGCCAGTATTTATTATTTTTC |
| v302 | 15 | GATCTAGAGTCGCGGCCcctattaCTTATACAGTTCATCCATTCCCATCACGTCG |
| v303 | 16 | GCCACCATGGTGTCGAAGGGCGAGGAGGAC |
| v304 | 17 | CTTCGACACCATGGTGGCGACCGGTG |
| v308 | 18 | CTTTCGATCGCCGCAGacgtcgtaaggctagttttgagaaggatcgcttgtctgggcaag |
| v309 | 19 | ATGTATCttaAAGCTTATCGATACGCGTACgctagccaaggtgctacgaaatccgttgtg |
| v353 | 20 | gttcacaacggatttcgtagcaccttggctagcGgctcctactcaaatacaaaaacatca |
| | | aattttctgtcaataaagcatatttatttatatttattttacaggaaagaattcctttta |
| | | aagtgtattttaacctataatgaaaaacgattaaaaaaaatacataaaataattcgaaaa |
| | | tttttgaatagcccaggttgataaaaattcatttcatacgttttataacttatgccccta |
| | | agtattttttgaccatagtgtttcaattctacattaattttacagagtagaatgaaacgc |
| | | cacctactcagccaagaggcgaaaaggttagctcgccaagcagagagggcgccagtgctc |
| | | actactttttataattctcaacttctttttccagactcagttcgtatatatagacctatt |
| | | ttcaatttaacgtcGGCATCCAAGTATCGCCATCGTTTTAGAGCTAGAAATAGCAAGTTA |
| | | AAATAAGGCTAGTCCGTTATCAACTTGAAAAAGTGGCACCGAGTCGGTGCtttttttgcc |
| | | tacctggagcctgagagttgttcaataaaataaaaatgtttcgtttttttgctttcgcca |
| | | gtatttattatttttcatcaatatgtattcgctagcGTACGCGTATCGATAAGCTTtaaG |
| | | ATAC |
| v357 | 21 | acgtcgtaaggctagttttgagaaggatcgcttgtctgggcaag |
| v358 | 22 | ctcaaaactagccttacgacgtGGCGATACTTGGATGCCCTGCGGCGATCGAAAGGCAAG |
| v359 | 23 | ATCCCCGGGCGAGCTCGcatatATCCGGGATGCGACTGCTCAATG |

### Generation of Transgenic Lines

CopyCat constructs were sent to Rainbow Transgenic Flies, Inc. for injection and were injected together with a Cas9-expressing plasmid (pBSHsp70-Cas9 was a gift from Melissa Harrison & Kate O'Connor-Giles & Jill Wildonger [Addgene plasmid #46294; RRID: Addgene_46294]) at a total DNA concentration of 1 ug/ul. The w2 and w5 constructs were injected into Oregon-R wildtype strain; the y1 and e1 constructs were injected into the isogenized Oregon-R strain carrying the w^{-EX1} mutation (small frameshift mutation in the first exon of white), giving them white eyes to make screening for transformants easier. G₀ larvae were sent back to us and were allowed to develop. The adult flies were crossed to each other in pools of 3-5 males with 3-5 females. The resulting G₁ flies were then screened for green fluorescence in their eyes as a marker of transgene insertion. Homozygous lines were constructed from single transformants crossed to wildtype (Oregon-R for w2 and w5, Oregon-R with w^{-EX1} for y1 and e1) flies of the opposite sex and following the recessive eye/body color phenotype and fluorescent marker in subsequent generations. Correct transgene insertion in each stock was confirmed by PCR and Sanger sequencing.

### Fly Rearing and Crosses

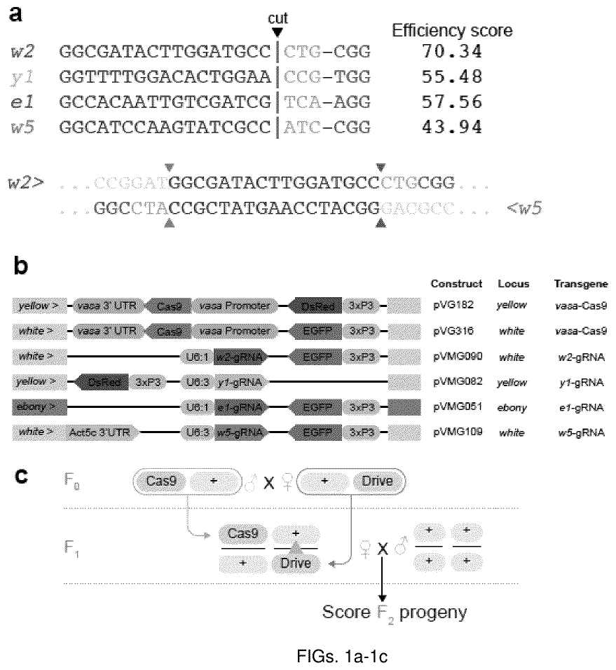

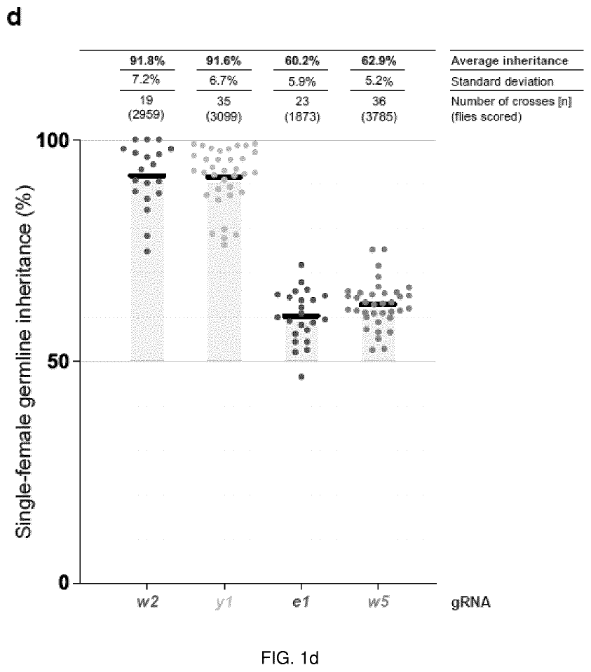

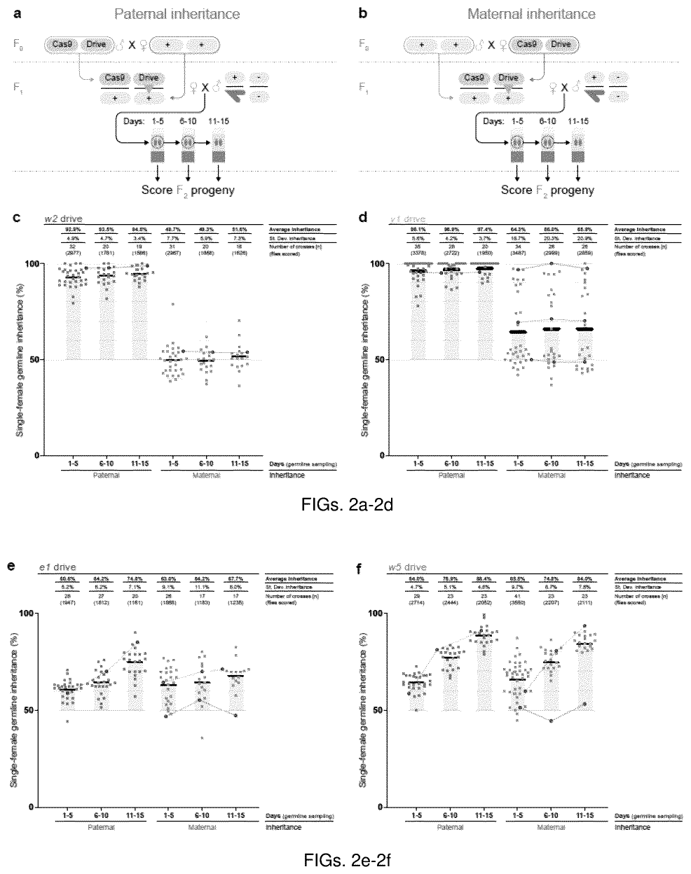

Flies were kept on standard cornmeal food with a 12/12 hour day/night cycle. Fly stocks were kept at 18° C. and all experimental crosses and cages were kept at 25° C. Flies were anesthetized with CO₂ in order to score phenotypes and make crosses. For all experimental crosses, virgin females were collected as pupae, then crossed the same day they eclosed. F₀ crosses were made in small pools of 3-5 males crossed to 3-5 females. F₁ crosses were made in single pairs and left for 5 days before discarding (FIGS. 1a-1d) or passing to a new vial (FIGS. 2a-2f). After allowing 18 days from the start of the cross for development, F₂ flies were scored for sex, body/eye color, and fluorescence (DsRed and/or GFP) as a marker of transgene inheritance using a Leica M165 F₂ Stereomicroscope with fluorescence. All gene drive experiments were performed in a high-security ACL1 (Arthropod Containment Level 1) facility dedicated to gene drive research in the Division of Biological Sciences at the University of California, San Diego. Crosses were made in shatter-proof polypropylene vials (Genesee Scientific Cat. #32-120) and all flies and vials were frozen for 48 hours before being removed from the facility, autoclaved, and discarded as biohazardous waste.

### Caged Population Protocols

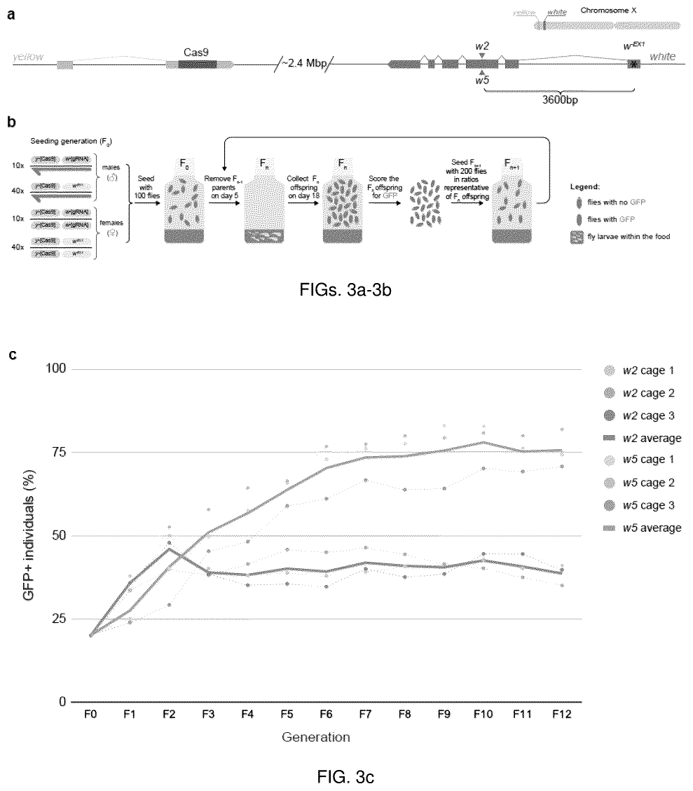

Caged Population Experiments in FIGS. 3a-3c:

For each w2 or w5 sample, bottles were seeded with 100 flies consisting of: 1) 40 Cas9, w^{-EX1} virgin females, 2) 40 Cas9, w^{-EX1} males, 3) 10 Cas9, w2 or w5-CopyCat virgin females, 4) 10 Cas9, w2- or w5-CopyCat males. The experimental outline is also shown in FIG. 3b. The Cas9 was expressed with a vasa promoter and marked with DsRed fluorescence, while the gRNAs were expressed with a U6 promoter and marked with GFP fluorescence in the eye. Each condition was performed in triplicate. After seeding each bottle, the crosses were left for 5 days before removing the parents. The remaining eggs and larvae remained in the 25° C. incubator until day 18, when all the offspring were phenotypically scored as male/female and for GFP fluorescence, with the fluorescent marker being indicative of CopyCat presence. Each following generation was seeded with a total of 200 flies with the same phenotypic ratios (male/female and GFP+/GFP−) observed in the entire offspring. All flies were kept on a standard cornmeal food with a 12/12-hour day/night cycle at 25° C. Experiments were executed using shatter-proof polypropylene bottles (Genesee Scientific Cat #: 32-129F) within the high-security ACL1 facility, providing the necessary safeguards for gene drive experiments.

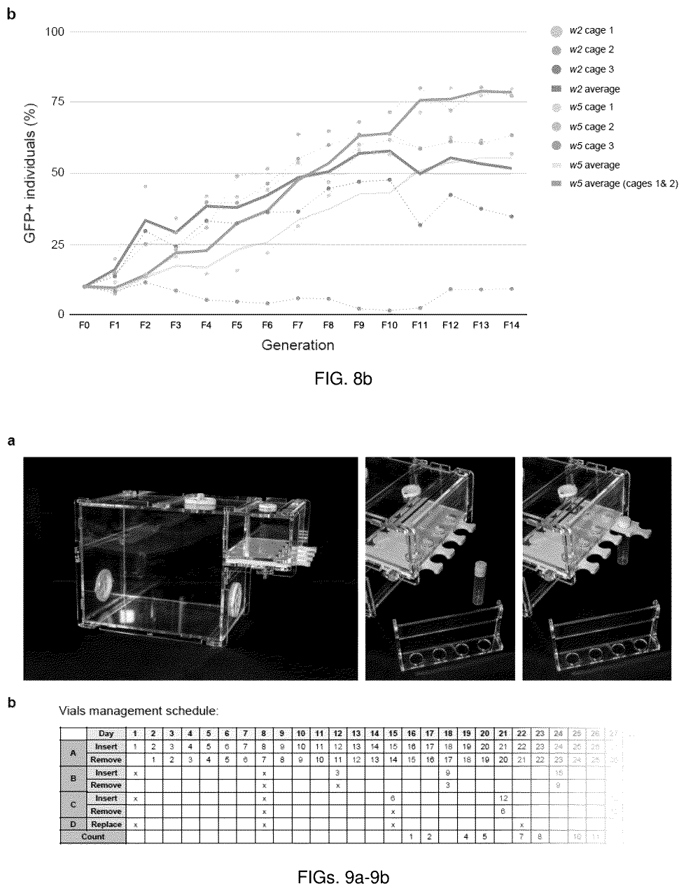

Caged population experiments in FIGS. 8a-8b:

For each w2 or w5 sample, bottles were seeded with 100 flies consisting of: 1) 50 Cas9, w^{-EX1} females, 2) 40 Cas, w^{-EX1} males, and 3) 10 Cas9, w2/w5-gRNA CopyCat virgin females, each marked the same promoter and fluorescent markers as used previously. Each condition was performed in triplicate. After seeding, parental flies were removed from the bottles on day 5, and the remaining larvae were left to develop until day 18. In this experiment, approximately 200 of the offspring were randomly sampled to seed the next population while remaining flies were scored for the GFP fluorescence, with the fluorescent marker being indicative of transgene inheritance. The bottle experiments were conducted on this schedule for 14 generations. In line with previous experiments, all flies were kept on a standard cornmeal food with a 12/12-hour day/night cycle at 25° C., and experiments were executed using shatter-proof polypropylene bottles (Genesee Scientific Cat #: 32-129F) within the same ACL1 facility.

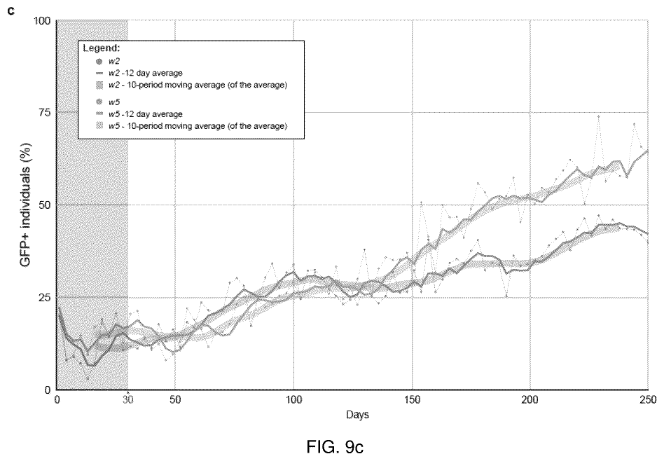

Overlapping-Generations Caged-Population Experiments in FIGS. 9a-9c:

For each w2 and w5 construct we seeded one large population cage with 300 flies consisting of: 1) 150 Cas9, w^{-EX1} virgin females, 2) 120 Cas9, w^{-EX1} males, and 3) 30 Cas9, w2- or w5-CopyCat males. Each of the two cages has four slots that can hold a single plastic vial for either population replenishment or feeding. The four slots are termed A, B, C and D. After initial seeding all slots were filled with fresh food vials, and after that they were handled with the following vial-swapping schedule: Vial A: was changed every day to collect eggs deposited by the population on a given day, after removal these vials were incubated at until ready for counting or population replenishment; any flies trapped in the vial after its removal were anesthetized and put back in the population so not to artificially remove individuals. Vial B/C: these two slots were used to replenish the population with offspring flies. Every third vial removed from slot A was incubated at 25° C. for 9 days and then added to either slot B or C and left there for 6 days to allow pupated flies to eclose and join the population. After 6 days, as most pupae had eclosed, the vials were discarded and substituted with the next replenishment vial in line. Vial D: this slot was used to keep a feeder vial which was replaced every 7 days so as not to allow a full developmental cycle to occur. The full vial swapping schedule is outlined in FIGS. 9a-9b. Both cages and vials were kept on a standard cornmeal food with a 12/12-hour day/night cycle at 25° C., and experiments were executed using shatter-proof polypropylene vials (Genesee Scientific Cat. #32-120) within the same ACL1 facility.

### Graph Generation and Statistical Analysis

All graphs were generated using GraphPad Prism 8, Google Sheets and modified in Adobe Illustrator. Statistical analyses were performed using GraphPad Prism 8. Kolmogorov-Smirnov tests were performed to test for normal distributions. Depending on those results, an unpaired t test (w2, e1) or a Mann Whitney test (y1, w5) was used to compare maternal vs. paternal inheritance for each drive element (FIGS. 2a-2f). To analyze how inheritance rates changed over time, a one-way ANOVA (w2, e1) or a Kruskal-Wallis test (y1, w5) was used. If a significant difference was detected here, a Dunn's multiple comparisons test (w5) or a Turkey's multiple comparisons test (e1) was used to determine statistically significant differences between timepoints (FIGS. 2a-2f). To compare split vs. maternal inheritance of the e1 drive with conversion in the male, an unpaired t-test was used (FIG. 8c). To analyze inheritance rates over time, a Kruskal-Wallis test followed by a Dunn's multiple comparisons test was used (FIG. 8c).

### Example 2

### Generation of Split Gene-Drive Systems with Diverse Efficiencies

To identify gRNAs with different efficiencies to attenuate the maternal effect caused by Cas9/gRNA deposition in the egg, four split, RNA-only gene-drive constructs were built, hereon referred to as “CopyCat” elements (FIGS. 1a-1d). As a positive control, two previously validated and highly efficient gRNAs, y1 and w2, that produce a strong maternal effect were included (FIG. 1a)¹⁹. two less efficient gRNAs: e1-gRNA, which has lower gene-drive activity¹⁹, and w5-gRNA, which cuts at a comparable location to w2 (FIG. 1a′), but with predicted lower efficiency by the CHOPCHOP analysis tool²⁵ were also used (FIG. 1a). Each of these gRNAs targets one of three pigmentation genes in Drosophila melanogaster that produce visible recessive phenotypes: yellow (y) for a lighter body, white (w) for white eye instead of red, and ebony (e) for a darker body.

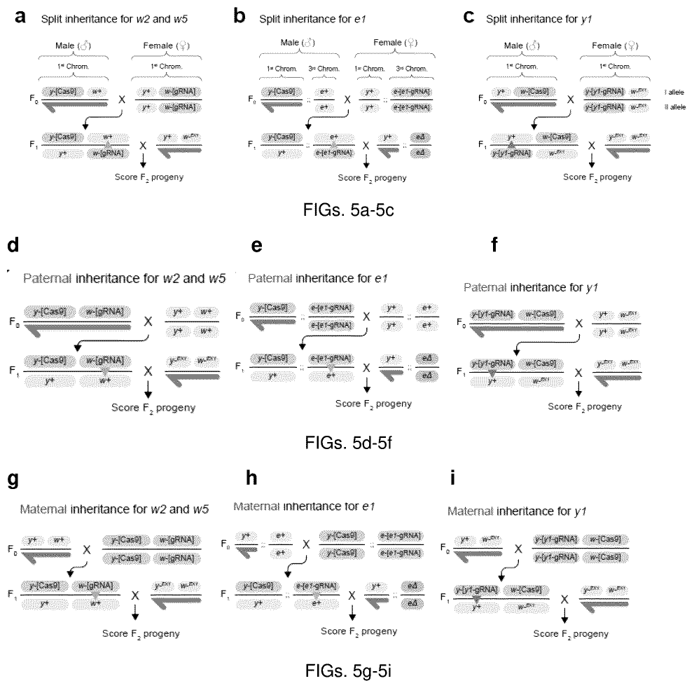

To evaluate the effectiveness of these elements in generating a gene drive, standard crosses were first performed without the confounding effect of a maternally inherited Cas9. To do this, male flies carrying a Cas9-expressing transgene were crossed with females from the generated CopyCat lines. From their F₁ progeny, trans-heterozygous virgin females that carried both Cas9 and CopyCat were collected. The females were then single-pair crossed with wildtype (Oregon-R) males (FIG. 1c; FIGS. 5a-5c) and the F₂ progeny from each cross was scored for the presence of fluorescent markers that tracked either the Cas9 or gRNA transgene. It was shown that the w2- and y1-gRNAs generated strong super-Mendelian inheritance of the CopyCat transgene, with 91.8% and 91.6% average inheritance rates, respectively. The e1-gRNA had a lower inheritance rate of 60.2% (FIG. 1d), in line with the previous work^{18,19}. The w5-gRNA also displayed a lower inheritance rate of 62.9%, comparable to that of the e1-gRNA (FIG. 1d), and in agreement with the activity predicted by the CHOPCHOP tool (FIG. 1a).

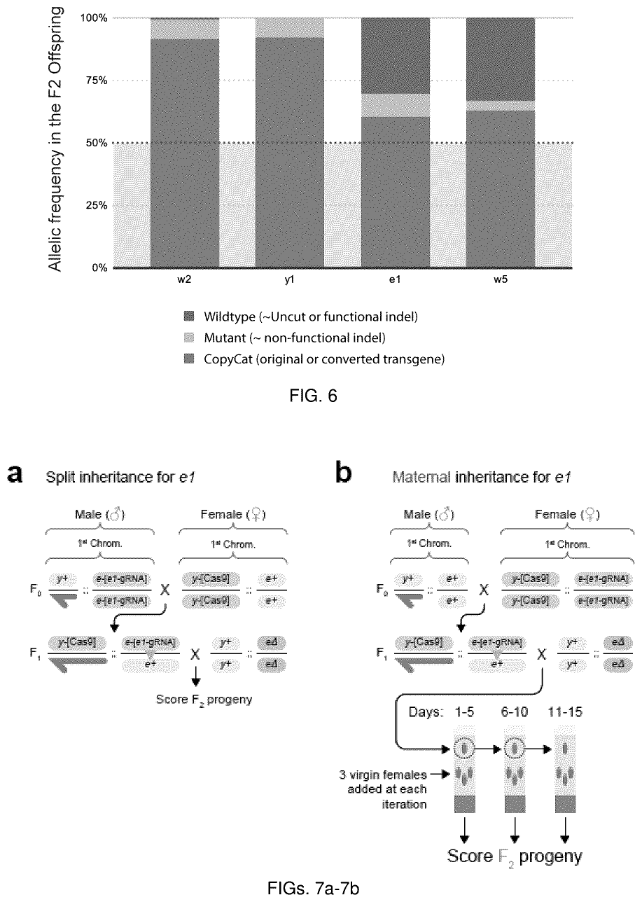

The lower gene-drive efficiency with e1- and w5-gRNAs could be due to either lower HDR conversion efficiency at these sites or lower target cleavage, with the latter resulting in less opportunity at conversion. Since the F₁ test animals were crossed to mutant counterparts (FIG. 5a-5c), these two alternatives could be distinguished by analyzing the F₂ phenotypes. For only e1 and w5, a high number of animals displaying wildtype phenotype was observed, which suggested that these gRNAs inefficiently cut their target, leaving several wildtype alleles in the F₁ germline. As a result, they were transmitted to the F₂ offspring without alteration (FIG. 6). These results confirmed previous observations for the activity of these gRNAs^{18,19} and identified w5 as an additional low-cutting gRNA. Thus, these gRNAs could potentially bypass maternal effects by delaying cutting beyond early development.

### Example 3

### Low-Efficiency gRNAs can Overcome Maternal Effects

To next evaluate the maternal effects of the four gRNA constructs, genetic crosses were first performed to combine each CopyCat element with a Cas9-expressing transgene to establish fruit fly lines that are homozygous for both transgenes. The paternal (FIG. 2a) or maternal (FIG. 2b) transgene inheritance were tested by crossing animals from these lines with wildtype flies (Oregon-R). From their F₁ progeny, heterozygous virgin females were then collected and they were single-pair crossed to mutant lines for the targeted locus (FIGS. 5a′-5c′ for paternal genetic crosses and FIGS. 5a″-5c″ for maternal ones). The inheritance for each of these crosses was evaluated by scoring the F₂ progeny for the presence of the fluorescent markers that tracked the Cas9 and gRNA transgenes. A strong maternal effect was observed with the w2-(FIGS. 2c) and y1-drive constructs, with a significant decrease in inheritance for both w2 and y1 (see columns 1 and 4 in FIGS. 2c and 2d, respectively; w2, p<0.0001; y1, p<0.0001). In the paternal condition, the w2-drive has ˜93% inheritance. However, only ˜50% drive inheritance was observed when the two constructs were inherited from the FO female in congruence with previous observations 19. A similar analysis was performed for the low-cutting gRNAs and no significant difference between the inheritance from an F₀ father or F₀ mother was observed (e1, p=0.2285; w5, p=0.1712). For the e1 construct, ˜61% and ˜63% (FIG. 2e) paternal and maternal inheritance were observed, respectively. The analogous values for the w5-gRNA construct were ˜64% and ˜66% (FIG. 2f) (columns 1 and 4 in FIGS. 2e and 2f). These results suggest that lower-cutting gRNAs can dampen the maternal effect generated by Cas9/gRNA deposition in the egg. The lower-efficiency gRNAs are also deposited in the egg along with Cas9, yet they would act in a delayed fashion cutting their DNA targets at later developmental stages in the germline where the HDR is the predominant DNA-repair pathway.

In the study of the separate transgene inheritance, in the F₂ offspring of the high-cutting w2- or y1-gRNAs, little to no w+ or y+ animals were observed in most instances, which suggests that close to 100% of the alleles were targeted and repaired by either HDR (gene-drive conversion) or NHEJ (indel). In contrast, when the drives were examined with low-cutting e1- or w5-gRNAs, a high number of F₂ animals that displayed the wildtype phenotype were observed. These results suggest that in the case of high-efficiency gRNAs, the cells of the flies hatched from pupae have already been fully exposed to gRNA activity. In contrast, in the low-efficiency cases, a considerable portion of cells were not yet targeted, suggesting that the germline could still be partially wildtype, and potentially prone to conversion in the adult.

### Example 4

### Low-Efficiency gRNAs Promote Progressive Allelic Conversion Over Time

In the routine tests of gene-drive performance, it was typically crossed the F₁ test individuals for 5 days, initiating the genetic cross after eclosion from the puparia to ensure healthy and successful crosses. In the e1 and w5 experiments, it was observed that the F₁ females still produced wildtype gametes, since it was observed wildtype animals in their offspring. Given that the Cas9 and gRNA transgenes were present in these F₁ females and were capable of expressing the two components in their germline, it may suggest further cutting and conversion would continue as the flies aged, to generate higher gene-drive inheritance. To test this suggestion, a subset of the F₁ crosses described above was taken and the flies were sequentially passed into two new food vials. the F₂ progeny produced by the same F₁ parents between days 6-10 and 11-15 since the initial setup were then evaluated (FIGS. 2a & 2b). It was observed that the average inheritance for the high-cutting w2 and y1 remained constant over time, suggesting that the germline of F₁ females is fully edited at eclosion from the puparia (FIGS. 2b & 2c). In contrast, when the progeny from the additional vials in the low-cutting e1 and w5 experiments were analyzed, a gradual increase of the average inheritance over time was observed in both the paternal and maternal lineages. For e1, the average inheritance progressed from 61% to 64% to 75% for the paternal lineage and from 63% to 64% to 68% for the maternal lineage over days 1-5, 6-10, and 11-15, respectively (FIG. 2e). For w5, the values rising from 64% to 77% to 88% for the paternal lineages and from 66% to 75% to 84% for the maternal lineages were observed (FIG. 2f). The w5 drive displayed a steeper increase relative to e1, given that the average inheritance increased by ˜10% for each additional 5 days that the F₁ flies were aged. The w5 drive also reached values comparable to the highly efficient y1- and w2-gRNAs when inherited through an F₀ male (˜85-95%, FIGS. 1a-1d and FIGS. 2a-2f) 16.19. Remarkably, in these experiments, almost all the values for single F₁ crosses increased over time for e1 and w5, in both the paternal and maternal inheritance (FIGS. 2e & 2f). Only a few F₂ vials that displayed a constant value of ˜50% were observed, indicative of the maternal effect (FIGS. 2e & 2f). This observation is further supported by the observation that the remaining animals lacking the CopyCat transgene always displayed the same phenotype (e+ or e−; and w+ or w−). These data suggest the occurrence of an early DNA cut that generated an indel allele that was inherited by all the non-drive progeny. These results reinforce the previously proposed model where the w2- and y1-gRNA usually cut to completion before the germline is established during early embryo development 19, and suggest that early embryo cutting can also happen when using low-cutting gRNAs, although to a much lower extent.

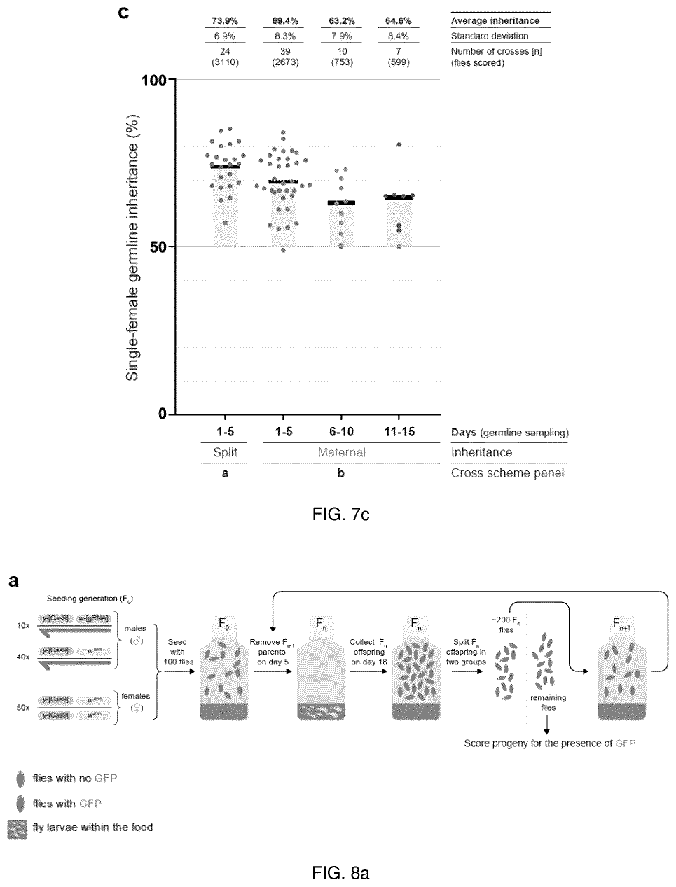

Given that all the previous results analyzed gene drive conversion in the female germline, whether progressive conversion over time, as observed in FIGS. 2a-2f, is a feature that is specific to the ovaries or if it could also occur in the testes. Since the e1-CopyCat is inserted on an autosome, it can also be used to evaluate the gene drive in the male germline. Thus, in both a split and conjunct maternal inheritance, the ability of the e1 construct to generate gene drive in males was test (FIGS. 7a-7b). When the offspring of F₁ male crosses of increasing age was evaluated, an average inheritance level that was similar to that of the split arrangement was observed, but the up-trend as seen for aging females was not observed (FIG. 7c). It is possible that developmental differences between the females' ovaries and the males' testes are responsible for the observed differences between the females' increasing gene drive conversion over time (FIG. 2e), and the constant inheritance values observed for males (FIG. 7c).

### Example 5

### The Low-Efficiency w5-gRNA Outperforms the Spread of w2 in Laboratory Caged Populations

Given that low-efficiency gRNAs seem to bypass the maternal effect and that progressive conversion in the adult germline can lead to high gene-drive rates, it was further tested whether these gene-drive constructs would spread more efficiently in a population. The two constructs targeting white were selected for three reasons: 1) both w2- and w5-gRNAs target essentially the same sequence (FIG. 1a′); 2) the phenotypic analysis would be identical, simplifying data interpretation; and 3) these constructs are inserted at the same X-linked location that ensures the gene drive only occurs in females, which should accentuate the effect of maternal Cas9/gRNA deposition on drive spread. Given that the constructs built here are split gene drives disrupting white and to avoid any potential fitness differences between the wildtype and drive individuals, it was used as the “wildtype” a white-mutant with a small indel in the first exon of the gene, which is far enough from the w2/w5 target site to avoid interference with the drive process (w^{-EX1}, FIG. 3a). Additionally, it was ensured that all individuals of the population would be homozygous/hemizygous for the transgene to avoid any potential fitness costs induced by the presence of the Cas9 source (FIG. 3b). To avoid sampling errors, a specific protocol was followed for seeding subsequent generations with 200 flies that are representative of the offspring (see above Methods and Materials, FIG. 3b).

This analysis was performed by seeding three bottles with 20% of flies that carried either the high-cutting w2-gRNA or the low-cutting w5-gRNA, following the protocol outlined in FIG. 3b to ensure no overlap between generations. It was observed that both the w2 and w5 constructs rapidly increased in frequency in the population within the first two generations, with w2 having slightly faster dynamics (FIG. 3c). Then, while the w2 drive stopped increasing in frequency and hovered around ˜40% after the F₂, the w5 drive continued to climb, plateauing around the F₁₀ generation at a frequency of ˜75% (FIG. 3c). The effects with w2 were attributed to the maternal-effect-driven generation of a high number of resistant alleles. These results suggest that while low-cutting gene drives exhibit lower drive conversion, since they bypass the maternal effect, they can display a net increase in performance.

This experiment was repeated to mimic the release of a putative mosquito gene-drive application and altered the protocol in 3 ways: 1) only male gene-drive animals (non-biting male mosquitoes are favored for wild releases) were released; 2) it was initially seeded with less flies (10% gene drive, or an effective 6.7%, as males carry one X-chromosome) to mimic an eventual release amount; and 3) sampling variability was included in the protocol to reflect random fluctuations in the wild (FIGS. 8a-8b). Similar to the previous experiment, it was observed that w2 seems to increase with faster dynamics, with an average prevalence of ˜52% in the population by the F₁₄ generation. Conversely, w5 increases at a slower pace, and two out of three populations reach ˜79% by the F₁₄ generation. An increase in gene-drive frequency (2-12% range) for one of the w5 cages was not observed, which may be due to sampling error between generations, as some of the bottles were presumably seeded with as little as 3 gene-drive flies (1.6% at generation F₁₀). This sampling error would also explain why the two other populations achieving higher drive prevalence appear to have an exponential increase at different generations (population 2, F₂₋₇; population 1, F₅₋₉). Therefore, as the failure of one of the w5 populations is likely due to innate sampling error in the protocol, an extension of the experiment into further generations could have eventually yielded a population with the same gene-drive prevalence as the other two. Separately, it was observed that the w2 drive reaches a higher average prevalence in the population (FIG. 8b) than in the previous experiment (FIG. 3c), which can be explained by two intertwined factors. First, in the previous experiment, the initial seeding was performed with both males and females which, for w2, should result in a high level of maternal-effect-driven generation of resistant alleles in the F₁. Second, it was previously seeded with a higher number of flies, which should lead to a faster accumulation of such resistant alleles, and thus prematurely stifle the w2-drive spread. While only two of the w5 populations displayed a successful spread of the gene drive in this experiment, they do so with comparable dynamics to those observed in FIG. 3c. These results further support the previous conclusion that the use of low-cutting gRNAs can yield a net increase in drive performance.

While the generational analysis of gene-drive spread allows for a simplified assessment of gene-drive performance, eventual releases in the wild would differ significantly from laboratory setups, due to factors such as lower population densities and overlapping generations. Therefore, the spread of the drives was evaluated in more wild-like conditions. Towards this goal, ad hoc laboratory cages were developed for easy sampling and maintenance of larger Drosophila melanogaster populations (˜1000 individuals) at low density, while ensuring no flies escaped during the experimental timeline, ensuring safe gene drive research while working with larger populations (FIG. 9a). To evaluate the spread of the w2 and w5 drives in these settings, two of these cages were seeded with 300 flies, 10% of which were gene-drive males. Following a daily, vial-swapping schedule (FIG. 9b and the above Methods & Materials section), the two populations were sampled every 3 days to generate an accurate picture of the gene drive progress over time. Also, given that the protocol feeds new offspring-flies into the cages every three days, little sampling bias were anticipated in this experiment. Similar to the observations in the previous experiments, w2 initially slightly outperformed w5 (up to day 120). Afterwards, the w5 drive increased its frequency to ˜75% prevalence by the experimental endpoint, while w2 reached only ˜40% (FIG. 9c). While this experiment was performed in similar conditions as described in FIGS. 8a-8b (i.e., seeding with 10% males, ˜250 days which is ˜14 generations), the two drives seem to have slightly slower spread dynamics. This effect is due to the older overlapping generations, which act as a constant dilution factor of drive prevalence. Nonetheless, these results further support the potential advantage of employing low-cutting gRNAs in future gene-drive strategies to bypass maternal effects for a net performance increase.

### Example 6

### A Model of the Observed Differences Between High- and Low-Cutting gRNAs

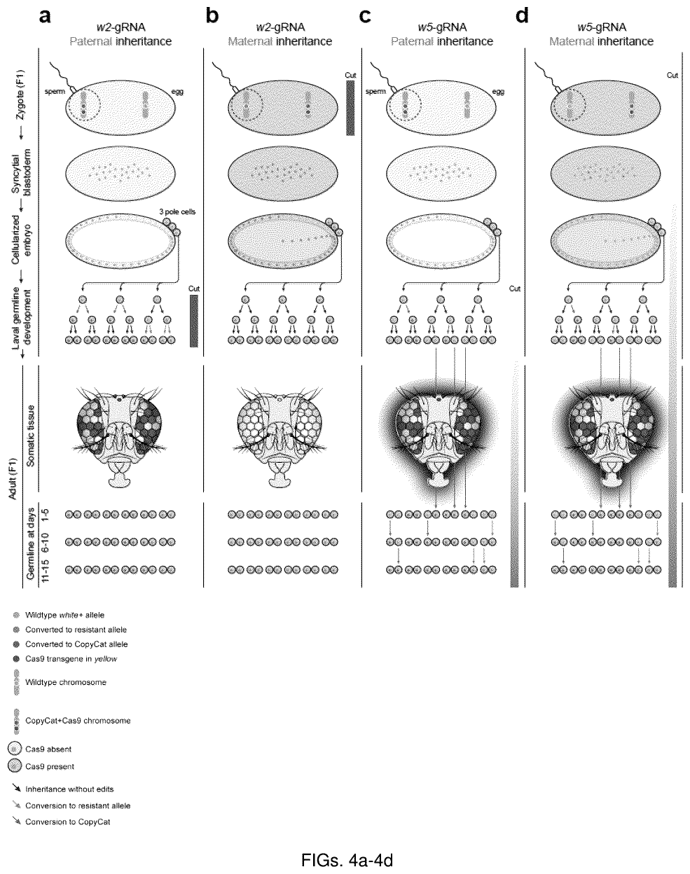

Based on the herein described results, a model was generated that explains all the observations for the drives targeting white (FIGS. 4a-4d). Consistently with previous work¹⁹, the w2-gRNA generates a strong gene drive when inherited from the father (FIG. 4a), while it generates a strong maternal effect when inherited from the mother (FIG. 4b). This phenomenon can be explained by the deposition of Cas9/gRNA complexes in the early embryo, which for high-cutting w2 can target the paternally inherited chromosome as early as the zygote (FIG. 4b)¹⁹. Furthermore, the phenotypic analysis of the F₁ animals reveals that while paternally inherited drives generate mosaic-eyed F₁ females (FIG. 4a), maternally inherited drives usually display a fully penetrant white-phenotype (FIG. 4b), in line with early targeting of the wildtype allele. In contrast, when analyzing the data from the low-cutting w5 experiments, mosaic animals were observed for both the paternal (FIG. 4c) and maternal (FIG. 4d) inheritance, suggesting later activity in both cases. While for w2 rapid and efficient cleavage occurs as soon as the Cas9 protein is produced, for w5 low levels of activity was observed throughout developmental stages (FIGS. 4c-4d). This is further supported by the increase in gene-drive inheritance observed when sampling the germline over time (FIGS. 2a-2f). Also, for w5 paternal inheritance, Cas9 is presented only after germline formation (FIG. 4c), in congruence with the observations for w2 (FIG. 4a). Moreover, rare cases of hampered inheritance for w5 were observed, which confirms that w5-gRNA/Cas9 complexes are also deposited in the egg, although here they can only rarely target the incoming paternal wildtype allele early enough to generate the maternal effect (FIG. 4d).

### REFERENCES

1. Scudellari, M. Self-destructing mosquitoes and sterilized rodents: the promise of gene drives. Nature vol. 571 160-162 (2019).
2. National Academies of Sciences, Engineering, and Medicine, Division on Earth and Life Studies, Board on Life Sciences & Committee on Gene Drive Research in Non-Human Organisms: Recommendations for Responsible Conduct. Gene Drives on the Horizon: Advancing Science, Navigating Uncertainty, and Aligning Research with Public Values. (National Academies Press, 2016).
3. James, A. A. Gene drive systems in mosquitoes: rules of the road. Trends Parasitol. 21, 64-67 (2005).
4. Gantz, V. M. et al. Highly efficient Cas9-mediated gene drive for population modification of the malaria vector mosquito Anopheles stephensi. Proc. Natl. Acad. Sci. U.S.A 112, E6736-43 (2015).
5. Adolfi, A. et al. Efficient population modification gene-drive rescue system in the malaria mosquito Anopheles stephensi. Nat. Commun. 11, 5553 (2020).
6. Carballar-Lejarazú, R. et al. Next-generation gene drive for population modification of the malaria vector mosquito, Anopheles gambiae. Proc. Natl. Acad. Sci. U.S.A 117, 22805-22814 (2020).
7. Hammond, A. et al. A CRISPR-Cas9 gene drive system targeting female reproduction in the malaria mosquito vector Anopheles gambiae. Nat. Biotechnol. 34, 78-83 (2016).
8. Kyrou, K. et al. A CRISPR-Cas9 gene drive targeting doublesex causes complete population suppression in caged Anopheles gambiae mosquitoes. Nat. Biotechnol. 36, 1062-1066 (2018).
9. Hammond, A. et al. Gene-drive suppression of mosquito populations in large cages as a bridge between lab and field. Nat. Commun. 12, 4589 (2021).
10. Courtier-Orgogozo, V., Morizot, B. & Boëthe, C. Agricultural pest control with CRISPR-based gene drive: time for public debate. EMBO Rep. 18, 878-880 (2017).
11. Teem, J. L. et al. Genetic Biocontrol for Invasive Species. Front Bioeng Biotechnol 8, 452 (2020).
12. Harvey-Samuel, T. et al. Culex quinquefasciatus: status as a threat to island avifauna and options for genetic control. CABI Agriculture and Bioscience 2, 1-21 (2021).
13. Grunwald, H. A. et al. Super-Mendelian inheritance mediated by CRISPR-Cas9 in the female mouse germline. Nature 566, 105-109 (2019).
14. Weitzel, A. J., Grunwald, H. A., Levina, R. & Gantz, V. M. Meiotic Cas9 expression mediates genotype conversion in the male and female mouse germline. bioRxiv (2021).
15. Godwin, J. et al. Rodent gene drives for conservation: opportunities and data needs. Proc. Biol. Sci. 286, 20191606 (2019).
16. Gantz, V. M. & Bier, E. Genome editing. The mutagenic chain reaction: a method for converting heterozygous to homozygous mutations. Science 348, 442-444 (2015).
17. Gantz, V. M. & Bier, E. The dawn of active genetics. Bioessays 38, 50-63 (2016).
18. López Del Amo, V. et al. Small-Molecule Control of Super-Mendelian Inheritance in Gene Drives. Cell Rep. 31, 107841 (2020).
19. Lopez Del Amo, V. et al. A transcomplementing gene drive provides a flexible platform for laboratory investigation and potential field deployment. Nat. Commun. 11, 352 (2020).
20. Feng, X. et al. Optimized CRISPR tools and site-directed transgenesis towards gene drive development in Culex quinquefasciatus mosquitoes. Nat. Commun. 12, 2960 (2021).
21. Champer, J. et al. Novel CRISPR/Cas9 gene drive constructs reveal insights into mechanisms of resistance allele formation and drive efficiency in genetically diverse populations. PLOS Genet. 13, e1006796 (2017).
22. Hammond, A. et al. Regulating the expression of gene drives is key to increasing their invasive potential and the mitigation of resistance. PLOS Genet. 17, e1009321 (2021).
23. Terradas, G. et al. Inherently confinable split-drive systems in Drosophila. Nat. Commun. 12, 1480 (2021).
24. Campos-Ortega, J. A. & Hartenstein, V. The Embryonic Development of Drosophila Melanogaster. (Springer-Verlag, 1985).
25. Labun, K. et al. CHOPCHOP v3: expanding the CRISPR web toolbox beyond genome editing. Nucleic Acids Res. 47, W171-W174 (2019).
26. Kandul, N. P. et al. Transforming insect population control with precision guided sterile males with demonstration in flies. Nat. Commun. 10, 84 (2019).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A method for improving gene drive effectiveness comprising using low-cutting gRNAs to bypass maternal effect that affects gene drive.

2. The method of claim 1, wherein said low-cutting gRNAs delays target cleavage to later in development by delaying drive activity.

3. The method of claim 1, wherein said low-cutting gRNAs are capable of continuing gene-drive conversation in an adult germline to achieve inheritance rates produced by high-cutter gRNAs.

4. The method of claim 1, wherein said low-cutting gRNAs has consistently improved gene drive performance.

5. The method of claim 1, wherein said method is used to modify or suppress wild populations of insects, animals, humans, and plants.

6. The method of claim 5, wherein said insects are flies or mosquitoes.

7. The method of claim 1, wherein gene drive is CRISPR gene drive.

8. The method of claim 7, wherein CRISPR action is delayed.

9. The method of claim 8, wherein the CRISPR action comprises homology-directed repair (HDR) as a predominant DNA-repair mechanism.

10. The method of claim 9, wherein HDR-mediated allelic conversion keeps acting in adult stages, resulting in progressively increasing gene drive inheritance as aging.

11. The method of claim 7, wherein said method is used for a human therapy where needs long or delayed action by CRISPR to ensure a gradual or timely delivery of genetic changes.

12. The method of claim 7, wherein said method is for agriculture use to have CRISPR act at different plant developmental stages.

13. The method of claim 12, wherein the plant development stage is pollen fruit or flower formation, wherein genetic edit is beneficial or effective.
